(19) United States
(12) Patent Application Publication
(10) Pub. No.: US 2018/0011904 A1
(43) Pub. Date: Jan. 11, 2018

(54) SYSTEMS, METHODS, AND DEVICES FOR PROCESSING RESEARCH DATA TO IDENTIFY NEW RECORDS

(71) Applicant: William Scott Harten, Woods Cross, UT (US)

(72) Inventor: William Scott Harten, Woods Cross, UT (US)

(21) Appl. No.: 15/201,718

(22) Filed: Jul. 5, 2016

(51) Int. Cl. G06F 17/30; G06F 19/00

(52) U.S. Cl. CPC G06F 17/30477; G06F 19/322; G06F 17/30289; G06F 17/30867

(57) ABSTRACT

A system for identifying records that likely include information about new records or individuals includes a candidate component, a relationship component, and a marking component. The candidate component is configured to obtain information about a candidate record that includes information matching information of a target record. The target record is stored in a database that includes a plurality of records and relationships between the records. The relationship component is configured to determine that the candidate record includes a greater number of hierarchal relationships of a specific relationship type than the target record. The marking component is configured to mark one or more of the candidate record and the target record for review to indicate that a potentially new record or individual for the database has been identified.

### TECHNICAL FIELD

The present disclosure relates to genealogical research and more particularly relates to processing research data to identify missing or new records.

### BACKGROUND

Genealogical research is of great interest for many purposes including medical research, historical studies, or even personal or religions interests. As vast amounts of genealogical and family history data is being digitized and shared, processing resources and tools to assist genealogical research are helpful to efficiently and accurately connect historical data and other research data.

### BRIEF DESCRIPTION OF THE DRAWINGS

Non-limiting and non-exhaustive implementations of the present disclosure are described with reference to the following figures, wherein like reference numerals refer to like or similar parts throughout the various views unless otherwise specified. Advantages of the present disclosure will become better understood with regard to the following description and accompanying drawings where:

FIG. 1 is a schematic diagram illustrating an example system for genealogical research, according to one embodiment;

FIG. 2 is a schematic block diagram illustrating components of a new records system, according to one embodiment;

FIG. 3 illustrates a table including identifier information for a target record and a candidate record, according to one embodiment;

FIG. 4 illustrates an interface for displaying and filtering nodes or records in a family tree or genealogical database, according to one embodiment;

FIG. 5 an interface for comparing records, according to one embodiment;

FIG. 6 is a schematic flow chart diagram illustrating a method for detecting and marking records that likely include a new person or record, according to one embodiment; and

FIG. 7 is a block diagram depicting an example computing device or system consistent with one or more embodiments disclosed herein.

### DETAILED DESCRIPTION

Digitization of historical records, genealogical research websites, and the sharing of genealogical research between professional and amateur researchers has greatly increased the amount of available digital genealogical data. However, sifting through and reviewing such large amounts of data is generally difficult and requires significant data storage, computing resources, and human research time.

Applicants have recognized that, at times, genealogical research can be significantly accelerated by adding a new person to a database or tree that is not presently in the database or tree. Applicants have developed systems, methods, and devices for locating records that likely correspond to new individuals that are not represented in a database or tree. According to one embodiment, a system for identifying records that likely include information about new records or individuals includes a candidate component, a relationship component, and a marking component. The candidate component is configured to obtain information about a candidate record that includes information matching information of a target record. The target record is stored in a database that includes a plurality of records and relationships between the records. The relationship component is configured to determine that the candidate record includes a greater number of hierarchal relationships of a specific relationship type than the target record. The marking component is configured to mark one or more of the candidate record and the target record for review to indicate that a potentially new record or individual for the database has been identified.

In one embodiment, a target record corresponds to a person or group of related persons already identified in a database or genealogical tree. In one embodiment, identifiers of target person(s) or record(s) are used to search other databases or documents for information or records that might extend what we know about the target. Matches that result from this or other searches may then be deemed candidate records or individuals. A candidate record corresponds to a person or group of related persons identified in a historical record or research database. In one embodiment, matching may be performed on a specific set of target and candidate records to determine if they correspond to the one or more of the same individuals or group of individuals. Matching may include an analytical process that compares the identifiers of the target to the identifiers in the candidate resulting in a decision for each candidate whether the candidate's identity matches the target's identity. Example identifiers that may be compared include record identifiers, names, birth dates, death dates, marriage dates, related individuals, locations, or any other information. Matching can be an extremely computing intensive process and include “fuzzy” or approximate matching even if not all identifiers are present in both records or if identifiers are not exactly the same to account for variations in spelling, misspelling, handwriting, abbreviations, or the like which may occur in historical or other records.

In some cases, when target and candidate identities do not match a matching process may indicate that identities for records pertain to two separate persons, and the candidate person may added to the database. When they match the candidate identifiers are added to the target identifiers for the target record or person. Matching identifiers increase confidence in what is already known. Conflicting identifiers decrease confidence. Non-matching and non-conflicting identifiers extend what is known.

In one embodiment, matching or potential matching records may be obtained and provided by another party or system. For example, family research organizations or websites may have access to actual research data or historical records and thus may be situated for performing matching processes. The results of these matching processes may then be provided to a user or a system as disclosed herein for detecting of records that likely include information about a new person that could be added. In one embodiment, matching records or hints may be provided by a third party system to systems disclosed herein for processing. In one embodiment, the matches or hints provided by a third party system may be processed to determine whether any of those matches or hints likely pertain to individuals not currently in a database or tree. For example, a system may determine how many relationships there are for each relationship type for both a target and a candidate record. In one embodiment, systems, methods, and embodiments may determine that a candidate record that matches a target record and includes more relationships of any type likely includes information about a new person. For example, a greater number of relationships in a candidate record may indicate that at least one of those relationships likely includes a person or record not currently in a database or tree. In one embodiment, target and candidate records include multiple persons grouped by relationship type. Relationship types include for example parent, spouse, child, and/or other relationships.

In one embodiment, a system may filter hints to display or highlight only candidate records or target records where there is likely a new record or person. For example, the count of related persons of a given relationship type in the candidate record may be greater than the count of related persons of the same relationship type in the target record. By way of illustration, if the candidate record includes three children and the target record includes only two then the candidate record likely contains a child that is not in the target record. In this situation, the extra child relationship may satisfy a goal of finding a new person to add to the tree, and this is displayed to communicate the opportunity to the user.

Addition of new persons can be of particular help during dependent research. Genealogical research traditionally involves moving backward in time from descendants to ancestors. However, due to record keeping and/or lack of digitized information for previous generations, digital genealogical research often hits barriers where data about ancestors is scarce. Thus, genealogical researchers may find it difficult to continue making progress in a backwards (earlier in time) direction. In these cases, it can be productive to perform dependents research in which research is performed moving forward in time. For example, research for a specific individual may continue backwards in time until records become scarce and progress is slow or difficult, and then move forward in time down genealogical lines including a common ancestor. A user who is researching their own pedigree may be able to research ancestors back to about 1800s and then find it difficult to make progress. At this point, the user may then do research moving forward with descendants from a common ancestor (e.g., cousin lines). As individuals researched move forward in time, the records will likely get better and genealogical research may become more productive in finding new individuals or connections to existing trees or researched lines. As these new connections are made, progress backwards in time may also be accelerated as there are additional names or lines which may be more easily researched.

Embodiments of systems, methods, and devices disclosed herein may significantly reduce computing and human resources to locate new persons. For example, identifying records as likely including new records or individuals based on relationship count requires very little computing resources, no additional information resources, and can be extremely productive. By way of illustration, the amount of resources required to match identifiers in target and candidate records can be extremely memory and processor intensive when compared with counting a total number of each type of relationship. Additionally, the locating and identifying of new persons can significantly reduce the amount of time spent per additional name or individual found. In testing, Applicants have found that the number of records that need to be examined by a researcher in order to locate new records can be reduced by a factor of 10-30 or more. For example, a researcher may need to examine 1/10^{th} to 1/30^{th} as many candidate records, or fewer, in order to add new individuals to a tree, when compared to matching procedures. Thus, the reduction in computing and human research time can lead to significantly improved research systems.

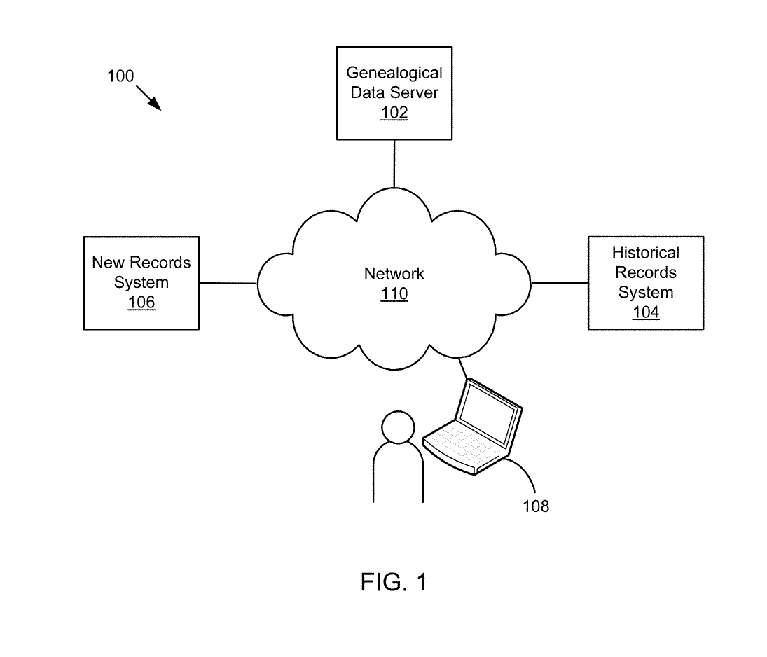

Turning now to the figures, FIG. 1 is a system 100 for genealogical research, according to one embodiment. The system 100 includes a genealogical data server 102, a historical records system 104, a new records system 106, and a user computer 108 that communicate over one or more networks 110, such as the Internet. The genealogical data server 102 may include or provide an online research system that allows devices, such as the user computer 108, or other online systems to view, search, and/or enter genealogical information. Example online research systems include those available from Ancestry.com®, FamilySearch.org®, or the like. The genealogical data server 102 may allow users to maintain, separate from other users or accounts, genealogical trees or databases that store information about individuals, relationships, or other genealogical information researched by the user.

The genealogical data server 102 may allow a user to search historical records, trees, or databases maintained by other users or entities to locate, connect, and/or include information into a database or tree managed by the user. In one embodiment, the genealogical data server 102 may perform automatic searching and/or matching services for identifying records or individuals that match or correspond to individuals already in a database or tree of the user. For example, the genealogical data server 102 may generate hints based on these matches which a user may review when accessing the genealogical data server 102 when doing genealogical research.

The historical records system 104 may include a system for accessing and/or storing historical records. Example historical records include governmental, religious, corporate, or private records that may be viewed or searched in digital form from remote devices, such as the user computer 108, or other online systems, such as the genealogical data server 102 and/or the new records system 106. By way of illustration, the types of historical records stored, managed, or searchable by the historical records systems 104 include census, marriage, birth, death, news, journal, or other records or information.

The new records system 106 is configured to process locate and/or identify records or information that corresponds to individuals who are likely not currently in a user's database or tree. For example, the new records system 106 may process data within a database, tree, or historical record stored or managed by the genealogical data server 102, historical records system 104, or user computer 108 to identify candidate records that have a greater number of relationships of a specific type than a target record.

The user computer 108 may include any type of computing or communication device. For example, the user computer 108 may include a smart phone, tablet, laptop, desktop, or other type of computer. The user computer 108 may access services, websites, or software provided by the genealogical data server 102, the historical records system 104, and/or the new records system 106 via a browser or application installed on the user computer 108. In one embodiment, the user computer 108 receives data and/or computer instructions from the genealogical data server 102, the historical records system 104, and/or the new records system 106 for processing and/or execution.

Although the genealogical data server 102, the historical records system 104, the new records system 106, and the user computer 108 are shown as separate devices or entities, embodiments may include a single computing device, server, or the like that includes two or more of the genealogical data server 102, the historical records system 104, the new records system 106, and the user computer 108. In one embodiment, for example, single system or server may include or implement the functionality of the genealogical data server 102, the historical records system 104, and the new records system 106. In another embodiment, the genealogical data server 102 may include the new records system 106.

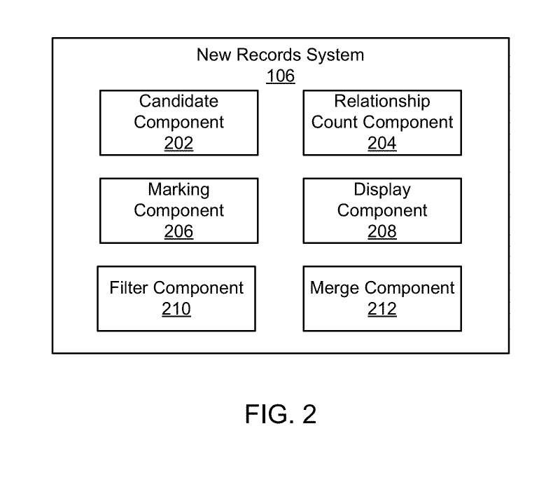

FIG. 2 is a schematic block diagram illustrating components of a new records system 106, according to one embodiment. The new records system 106 includes a candidate component 202, a relationship component 204, a marking component 206, a display component 208, a filter component 210, and a merge component 212. The components 202-212 are given by way of example only and may not all be included in all embodiments. For example, each of the components 202-212 may be included in or may be implemented as part of a separate device or system.

The candidate component 202 is configured to obtain information about a candidate record that may match a target record in a tree or database. For example, the candidate record may include information matching information of the target record, such as a name, birth or death date, or a relationship with another individual. The candidate record may be obtained by matching the candidate record with the target record using a matching procedure. In one embodiment, the candidate component 202 receives an indication that the candidate record likely corresponds to a same individual as the target record. This indication may be provided by another system (such as the genealogical data server 102) or a user. In one embodiment, the candidate component 202 receives the candidate record in response to a matching operation matching one or more values or parameters of the candidate record with the target record. The matching operation may be performed or automated by the new records system 106, a genealogical data server 102, a historical records system 104, or a user computer 108. Once matching procedure determines a likely match, the target record or candidate record may be flagged, tagged, or marked to indicate that a likely match exists and should be reviewed by a human researcher. The candidate record or other indication may be provided to a user or to the candidate component 202 as a hint.

The candidate record may include data obtained from a historical document, a family tree database, or any other location. The candidate record may include identifying information used to determine an identity of a user. For example, the candidate record may include a name, birth date, death date, marriage date, location, spouse name, child name, parent name, occupation, event descriptions involving a person, or the like. Example types of records which may be used as candidate records include a historical record, a family history database, a genealogical tree, or other information that is not in a user's or target tree or database. In some embodiments, only a sub portion of this data is available as gaps in data often exist in historical documents or genealogical research databases.

Similarly, the target record may include identifying information for an individual or their relations. The target record may include a record that is included in a database or family tree of a user or researcher. The database or tree may include a plurality of records and relationships between the records. For example, a record may correspond to a specific individual and relationships between the records may indicate a parent, child, or marriage relationship between the individuals.

The relationship component 204 is configured to determine a number of relationships for a candidate record and/or a target record. For example, the relationship component may count a number of relationships for each relationship type. In one embodiment, counts for a parent relationship type, child relationship type, and/or a marriage relationship type may be computed for both a target record and a corresponding candidate record. A relationship may include a hierarchical relationship in that relationships may include a direction as well as connections with two individuals (or records).

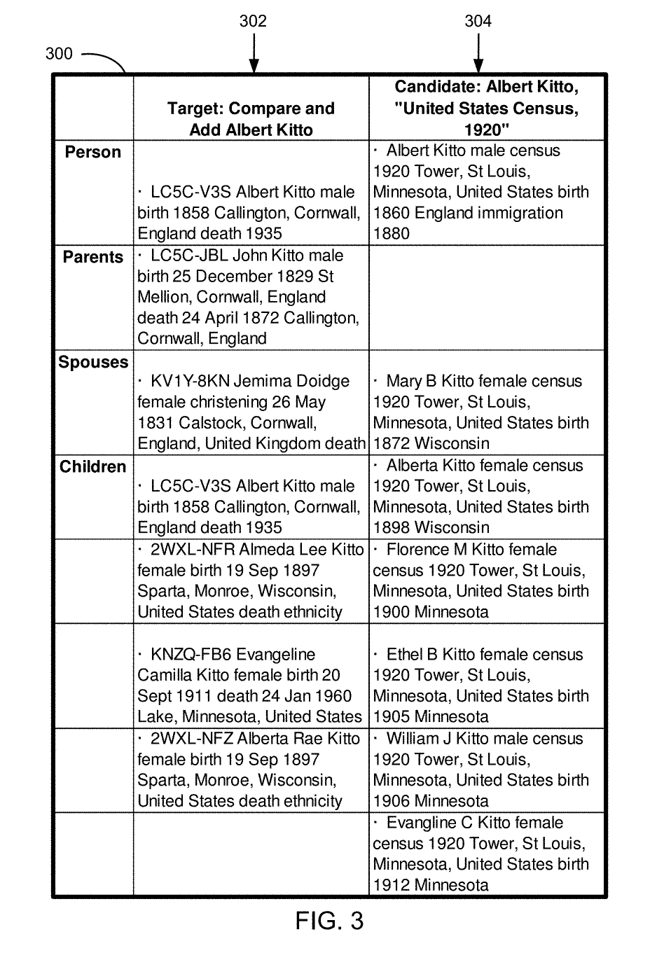

FIG. 3 illustrates a table 300 including identifier information for a target record 302 and a candidate record 304. As discussed previously, the target record 302, and associated information, may already be include within a research database of a user or organization while the candidate record, and associated information, may be located in a different database or document. For example, each of the individuals in the target record 302 includes a unique identifier (e.g., see LC5C-V3S for Albert Kitto) for the target database or tree while information in the candidate records does not include similar identifiers. The candidate record 304 may be provided or obtained in response to a matching process that matches the candidate record 304 with the target record 302. For example, the matching process may match one or more values in the candidate record 304 with the target record 302. Based on the values that match and/or how closely they match, the candidate record 304 may be provided to the new records component as a hint or as a record that likely corresponds to the target record 302.

The relationship component 204 may count a number of each type of relationship. For example, the relationship component 204 may determine a number of parent relationships, spousal relationships, and/or children relationships for Albert Kitto in the target record 302 as well as for Albert Kitto in the candidate record 304. For the target record 302, the relationship component 204 may determine that there is one parent relationship (John Kitto), one spouse relationship (Jemima Doidge), and four child relationships (Albert Kitto, Almeda Lee Kitto, Evangeline Camilla Kitto, and Alberta Rae Kitto). For the candidate record 304, the relationship component 204 may determine that there are zero parent relationships, one spouse relationship (Mary B Kitto), and five child relationships (Albert Kitto, Almeda Lee Kitto, Evangeline Camilla Kitto, Alberta Rae Kitto, and Evangline C Kitto). Based on the relationship counts, the relationship component 204 may determine that there are more child relationships (5 children) for the candidate record 304 than the target record. The relationship component 204 may provide an indication that there are a greater number of relationships for the candidate record to the marking component 206 for processing and/or flagging, marking, or otherwise indicating that the candidate record 304 should be reviewed.

In one embodiment, the number of relationships is determined without comparing whether a specific relationship is the same relationship. For example, the target record 302 includes a single spouse relationship with Jemima Doidge while the candidate record 304 includes a single spouse relationship with Mary B. Kitto. The relationship component 204 may simply count the number of relationship without accounting for identification information for individuals on the other end of the relationship. Counting the number of relationships may be significantly less computationally expensive as matching process between the spouses (or other individuals) is not required. In one embodiment, the identifying information may be taken into account and thereby the relationship component 204 may identify that the relationship with Mary B Kitto constitutes a new record or relationship.

Returning to FIG. 2, the marking component 206 is configured to mark, flag, or otherwise create or record an indication that a candidate record likely includes a new record or new individual. In one embodiment, the marking component 206 may create an entry in a database corresponding to a target record referencing the candidate record as likely including information about a new individual not yet in the database. In one embodiment, the marking component 206 may mark one or more of the candidate record and the target record for review to indicate that a potentially new record for the database has been identified. For example, after logging into an account a user (such as a researcher) may be presented with one or more hints that reference records that likely include new records or information for a tree or database managed by the account. In one embodiment, the marking component 206 is configured to mark one or more of the candidate record and the corresponding target record by storing and associating an identifier with the target record in the database. For example, the marking component 206 may store data that indicates the target records is classified as a record having at least one candidate record that likely includes a new individual not present in the user's database or tree.

The marking component 206 may mark digital records to indicate that they should be reviewed by a user or presented to a user for review. The marking component 206 may enable the display component 208 or another component to, based on markings or other stored digital indicators, flags, or classifications, communicate or identify records of interest to a user. For example, a list of records marked as likely including a new individual may be presented to a user, an alarm or audible voice alert may be played, or a notification (email, text, or other message) may be sent to a user for all records or individuals that have been digitally marked as likely including information about a new individual or record.

The display component 208 is configured to display information about a user's tree or database. For example, the display component 208 may display a graphical representation of a database or tree for viewing by a user. In one embodiment, the graphical representation includes a graphical hierarchical tree with nodes and connections between the nodes. The nodes may each correspond to a different individual (or record for that individual) stored in the database. The connections between the nodes may indicate relationships (such as relationships) between records or individuals represented by the nodes. For example, the relationships may indicate a parent, child, or marriage relationship between individuals represented by different nodes.

In one embodiment, the display component 208 displays nodes based on a category or classification of nodes. For example, nodes with a first classification may be shown with a different shape, size, color, or like than a second classification. For example, a target record that has been matched with a candidate record may belong to a first classification, while a target record that has a corresponding candidate record that has more relationships of a specific type belongs to a second classification. Yet another classification may include records that do not have any corresponding candidate records. In one embodiment, the display component 208 causes a node corresponding to a target record that has a corresponding candidate record with more relationships of a specific type with a different color, shape, or other indicator than nodes without a corresponding candidate record with a greater number of relationships. In one embodiment, the different in how a node or record is displayed may indicate to a user that the user's review is helpful or required. For example, a target record that has a corresponding candidate record with more relationships may be displayed with brighter colors, in a larger side, with bolded text, or with other visual indicator that the target record (and/or candidate record) should be reviewed.

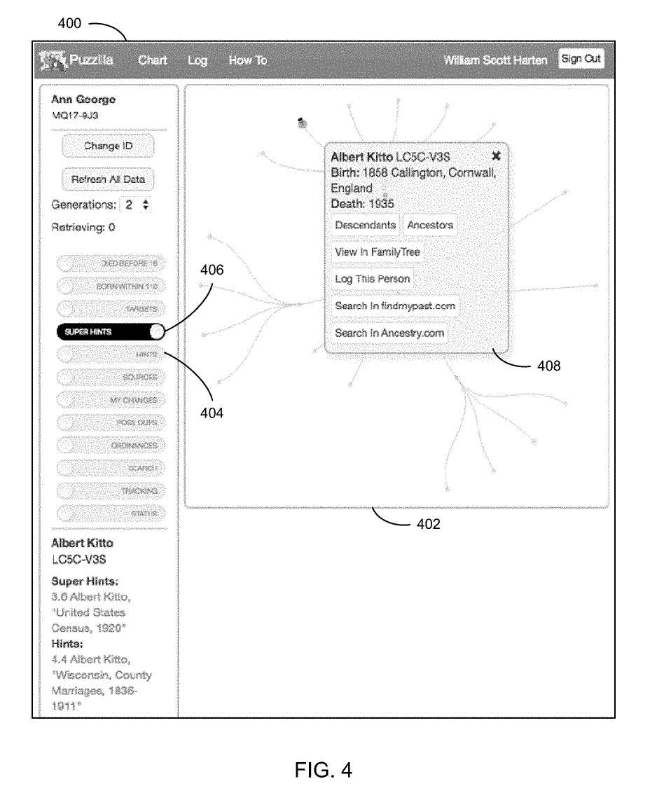

FIG. 4 illustrates an interface 400 for displaying nodes or records in a family tree or genealogical database provided by the display component 208. The interface 400 includes a graphical section 402 where nodes and connectors between nodes are displayed. The nodes may each correspond to an individual (or groups of individuals) in a family tree or database. The connectors may indicate a parent, child, or marriage relationship between the nodes.

The interface may include a plurality of selectors to filter the display of the nodes and/or connectors in the graphical section 402. For example, the selectors include a hints selector 404 which may be selected to show which nodes have corresponding hints. In one embodiment, the hints selector 404 may be used to show nodes that have a corresponding candidate record. For example, the hints selector 404 may show nodes that correspond to target records for which a candidate record has been identified using a matching process. In one embodiment, the selectors include a super hints selector 406 which may be used to show nodes that have a corresponding candidate record with more relationships than a target node corresponding to the node. For example, the super hints selector 406 may be used to show nodes for records that have been marked by the marking component 206. Thus, a user may be able to easily and quickly ascertain which records in a database have candidate records which likely include a new individual. If a user selects or hovers over a node, a pop-up 408 may be displayed showing detail for that specific node. The detail may include any information about a record corresponding to the node, such as name, birth, or other information. Additionally, the pop-up may include buttons or options which may be selected to display information about ancestors, descendants, or matching candidate records.

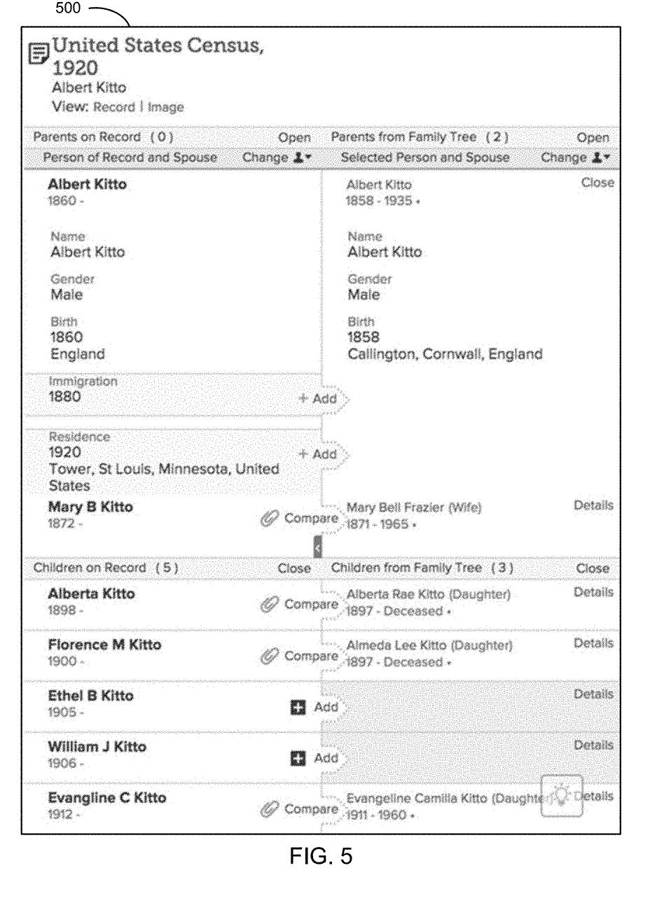

In one embodiment, the display component 208 displays a target record and a candidate record for review buy a user. For example, information about the candidate record and target record may be displayed side by side so that the user can determine what information should be merged into the user's database or tree. A user may be able to select a whole candidate record or portions or subsections of the candidate record for incorporation into the target record, tree, or database. In one embodiment, the display component 208 may display an option for a user to select that causes a browser to navigate to a different website (e.g., a website of a third party) to view the candidate and target record. For example, FIG. 5 illustrates an interface 500 for comparing records which may be displayed by third-party website, FamilySearch.org®, upon selection of a node or record.

Returning to FIG. 2, the filter component 210 is configured to filter information based on a classification, marking, or flag. For example, the filter component 210 may filter records, or nodes displayed in an interface so that the displayed records includes or omits records having different classifications, markings, or the like. In one embodiment, the filter component 210 is configured to present filter options to a user which may be selected to modify what is displayed. In one embodiment, the filter component 210 is configured to filter records in a database or tree based on the presence of an indication on a record that a potentially new record is in or associated with a corresponding candidate record. For example, a user may select the super hints selector 406 in FIG. 4 and see records or nodes which have corresponding candidate records having more relationships of a specific type than the record or node already in a tree or database. A user may be able to quickly see and review records that likely have new records and thereby significantly accelerate genealogical research.

The merge component 212 is configured to merge information from a candidate record into a tree, database, or target record. For example, after reviewing a candidate record, the user may select an option to add the candidate record, information from the candidate record, and/or a new record corresponding to a new individual to a tree, database, or target record. In one embodiment, the merge component 212 may merge or add the information by sending a message to a genealogical data server 102 to perform the actual modification or addition to a tree, database, or record.

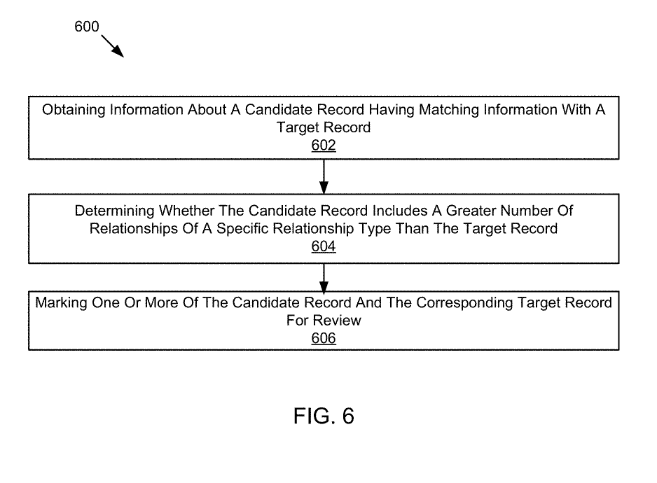

FIG. 6 is a schematic flow chart diagram illustrating an example method 600 for detecting and marking records that may include information about new individuals. The method 600 may be performed by a new records system, such as the new records system 106 in any of FIGS. 1-2.

The method 600 begins and a candidate component 202 obtains 602 information about a candidate record that includes information matching information of a target record. The target record is stored in a database comprising a plurality of records and relationships between the records. A relationship component 204 determines 604 whether the candidate record has a greater number of relationships of a specific relationship type than the target record. The marking component 206 marks 606 one or more of the candidate record and the corresponding target record for review to indicate that a potentially new record for the database has been identified. The marking component 206 may mark 606 one or more of the candidate record and the corresponding target record in response to the relationship component 204 determining 604 that the candidate record has a greater number of relationships of a specific relationship type than the target record.

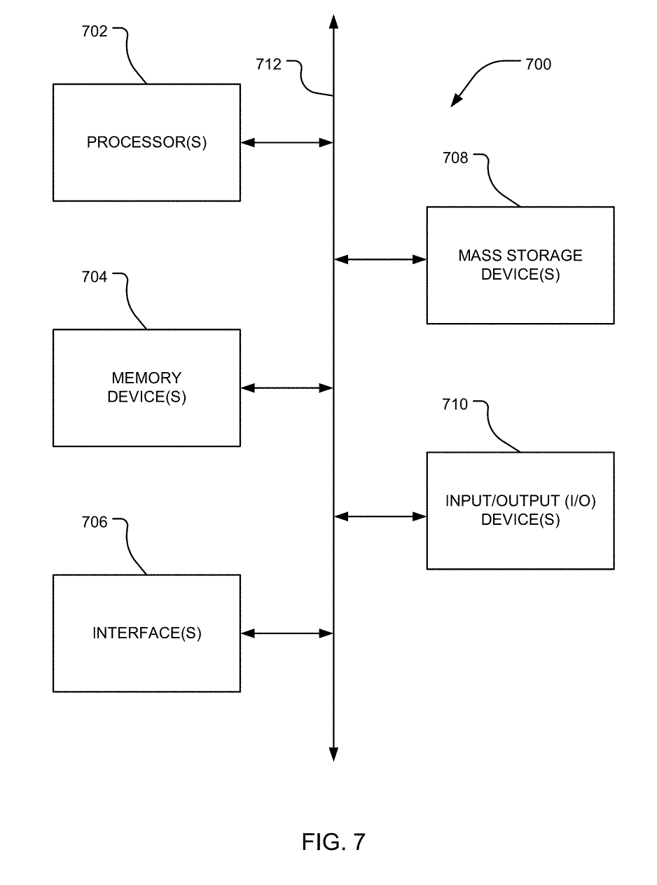

FIG. 7 is a block diagram depicting an example computing device 700. In some embodiments, computing device 700 is used to implement one or more of the systems and components discussed herein. Further, computing device 700 may interact with any of the systems and components described herein. Accordingly, computing device 700 may be used to perform various procedures and tasks, such as those discussed herein. Computing device 700 can function as a server, a client or any other computing entity. Computing device 700 can be any of a wide variety of computing devices, such as a desktop computer, a notebook computer, a server computer, a handheld computer, a tablet, and the like.

Computing device 700 includes one or more processor(s) 702, one or more memory device(s) 704, one or more interface(s) 706, one or more mass storage device(s) 708, and one or more Input/Output (I/O) device(s) 710, all of which are coupled to a bus 712. Processor(s) 702 include one or more processors or controllers that execute instructions stored in memory device(s) 704 and/or mass storage device(s) 708. Processor(s) 702 may also include various types of computer-readable media, such as cache memory.

Memory device(s) 704 include various computer-readable media, such as volatile memory (e.g., random access memory (RAM)) and/or nonvolatile memory (e.g., read-only memory (ROM)). Memory device(s) 704 may also include rewritable ROM, such as Flash memory.

Mass storage device(s) 708 include various computer readable media, such as magnetic tapes, magnetic disks, optical disks, solid state memory (e.g., Flash memory), and so forth. Various drives may also be included in mass storage device(s) 708 to enable reading from and/or writing to the various computer readable media. Mass storage device(s) 708 include removable media and/or non-removable media.

I/O device(s) 710 include various devices that allow data and/or other information to be input to or retrieved from computing device 700. Example I/O device(s) 710 include cursor control devices, keyboards, keypads, microphones, monitors or other display devices, speakers, printers, network interface cards, modems, lenses, CCDs or other image capture devices, and the like.

Interface(s) 706 include various interfaces that allow computing device 700 to interact with other systems, devices, or computing environments. Example interface(s) 706 include any number of different network interfaces, such as interfaces to local area networks (LANs), wide area networks (WANs), wireless networks, and the Internet.

Bus 712 allows processor(s) 702, memory device(s) 704, interface(s) 706, mass storage device(s) 708, and I/O device(s) 710 to communicate with one another, as well as other devices or components coupled to bus 712. Bus 712 represents one or more of several types of bus structures, such as a system bus, PCI bus, IEEE 1394 bus, USB bus, and so forth.

For purposes of illustration, programs and other executable program components are shown herein as discrete blocks, although it is understood that such programs and components may reside at various times in different storage components of computing device 700, and are executed by processor(s) 702. Alternatively, the systems and procedures described herein can be implemented in hardware, or a combination of hardware, software, and/or firmware. For example, one or more application specific integrated circuits (ASICs) can be programmed to carry out one or more of the systems and procedures described herein. As used herein, the terms “module” or “component” are intended to convey the implementation apparatus for accomplishing a process, such as by hardware, or a combination of hardware, software, and/or firmware, for the purposes of performing all or parts of operations disclosed herein.

### EXAMPLES

The following examples pertain to further embodiments.

Example 1 is a method for that includes obtaining information about a candidate record, wherein the candidate record includes information matching information of a target record. The target record is stored in a database comprising a plurality of records and relationships between the records. The method includes determining whether the candidate record comprises a greater number of hierarchal relationships of a specific relationship type than the target record. The method includes marking one or more of the candidate record and the corresponding target record for review to indicate that a potentially new record for the database has been identified.

In Example 2, obtaining information about the candidate record in Example 1 includes receiving an indication that the candidate record may correspond to a same individual or entity as the target record.

In Example 3, obtaining information about the candidate record in any of Examples 1-2 includes receiving the candidate record in response to a matching operation matching one or more values or parameters of the candidate record with the target record.

In Example 4, the database in any of Examples 1-3 includes a genealogical database in which the plurality of records correspond to individuals and the relationships correspond to a family relation between the individuals corresponding to the records.

In Example 5, the database in Example 4 includes a family tree corresponding to a user or account.

In Example 6, the specific relationship type in any of Examples 4-5 includes one or more of a parent relationship, a child relationship, or a marriage relationship.

In Example 7, marking in any of Examples 1-6 includes storing and associating an identifier with the target record in the database, wherein the identifier indicates that a potentially new record not present in the database is available in the candidate record.

In Example 8, marking in any of Examples 1-7 includes storing and associating information linking the candidate record with the target record in the database.

In Example 9, the method in any of Examples 1-8 further includes displaying a graphical hierarchical tree comprising nodes and connections between the nodes, wherein the nodes correspond to the plurality of records and the connections correspond to the hierarchical relationships. Marking the target record includes graphically marking a node corresponding to the target record.

In Example 10, the method in any of Examples 1-9 further includes filtering a plurality of target records comprising the target record based on the presence of an indication that a potentially new record is in or associated with corresponding candidate records.

In Example 11, the candidate record in any of Examples 1-10 includes one or more of: a historical record corresponding to a historical document; or a record in a second database separate from a first database, wherein the first database comprises the database.

Example 12 is a system that includes a candidate component, a relationship component, and a marking component. The candidate component is configured to obtain information about a candidate record, wherein the candidate record includes information matching information of a target record. The target record is stored in a database includes a plurality of records and relationships between the records. The relationship component is configured to determine that the candidate record includes a greater number of hierarchal relationships of a specific relationship type than the target record. The marking component is configured to mark one or more of the candidate record and the target record for review to indicate that a potentially new record for the database has been identified.

In Example 13, the candidate component in Example 12 is configured to obtain information about the candidate record by one or more of: receiving an indication that the candidate record likely corresponds to a same individual as the target record; or receiving the candidate record in response to a matching operation matching one or more values or parameters of the candidate record with the target record.

In Example 14, the database in any of Examples 12-13 includes a genealogical database wherein the plurality of records correspond to individuals and wherein the relationships correspond to a family relation between the individuals.

In Example 15, the specific relationship type in Example 14 includes one or more of a parent relationship, a child relationship, or a marriage relationship.

In Example 16, the marking component in any of Examples 12-15 is configured to mark one or more of the candidate record and the corresponding target record by storing and associating an identifier with the target record in the database, wherein the identifier indicates that information about a new individual not present in the database is likely available in the candidate record.

In Example 17, the system in any of Examples 12-16 further includes a display component configured to: display a graphical hierarchical tree comprising nodes and connections between the nodes, wherein the nodes correspond to the plurality of records and the connections correspond to the relationships; wherein the display component displays a node corresponding to the target record with a different color, shape, or other indicator than nodes without a corresponding candidate record with a greater number of relationships.

Example 18 is a computer readable storage media storing instructions that, when executed by one or more processors, cause the processors to obtain information about a candidate record, wherein the candidate record includes information matching information of a target record. The target record is stored in a database including a plurality of records and relationships between the records. The database comprises a genealogical database in which the plurality of records correspond to individuals and the relationships correspond to a family relation between the individuals. The instructions cause the one or more processors to determine whether the candidate record has a greater number of hierarchal relationships of a specific relationship type than the target record. The specific relationship type includes one or more of a parent relationship, a child relationship, or a marriage relationship. The instructions cause the one or more processors to mark one or more of the candidate record and the target record as part of a class for which a potentially new record for the database has been identified.

In Example 19, the instructions are configured to cause the processors to mark one or more of the candidate record and the target record in Example 18 by storing and associating an identifier with the target record in the database, wherein the identifier indicates that a potentially new record not present in the database is available in the candidate record.

In Example 20, the computer readable storage media in any of Examples 18-19 further includes instructions that cause the processors to displaying a graphical hierarchical tree comprises nodes and connections between the nodes, wherein the nodes correspond to the plurality of records and the connections correspond to the relationships. Marking the target record includes graphically marking a node corresponding to the target record.

Example 21 is an apparatus or system that includes means to perform a method or realize an apparatus or system as in any of Examples 1-20.

Various techniques, or certain aspects or portions thereof, may take the form of program code (i.e., instructions) embodied in tangible media, such as floppy diskettes, CD-ROMs, hard drives, a non-transitory computer readable storage medium, or any other machine readable storage medium wherein, when the program code is loaded into and executed by a machine, such as a computer, the machine becomes an apparatus for practicing the various techniques. In the case of program code execution on programmable computers, the computing device may include a processor, a storage medium readable by the processor (including volatile and non-volatile memory and/or storage elements), at least one input device, and at least one output device. The volatile and non-volatile memory and/or storage elements may be a RAM, an EPROM, a flash drive, an optical drive, a magnetic hard drive, or another medium for storing electronic data. One or more programs that may implement or utilize the various techniques described herein may use an application programming interface (API), reusable controls, and the like. Such programs may be implemented in a high-level procedural or an object-oriented programming language to communicate with a computer system. However, the program(s) may be implemented in assembly or machine language, if desired. In any case, the language may be a compiled or interpreted language, and combined with hardware implementations.

It should be understood that many of the functional units described in this specification may be implemented as one or more components, which is a term used to more particularly emphasize their implementation independence. For example, a component may be implemented as a hardware circuit comprising custom very large scale integration (VLSI) circuits or gate arrays, off-the-shelf semiconductors such as logic chips, transistors, or other discrete components. A component may also be implemented in programmable hardware devices such as field programmable gate arrays, programmable array logic, programmable logic devices, or the like.

Components may also be implemented in software for execution by various types of processors. An identified component of executable code may, for instance, comprise one or more physical or logical blocks of computer instructions, which may, for instance, be organized as an object, a procedure, or a function. Nevertheless, the executables of an identified component need not be physically located together, but may comprise disparate instructions stored in different locations that, when joined logically together, comprise the component and achieve the stated purpose for the component.

Indeed, a component of executable code may be a single instruction, or many instructions, and may even be distributed over several different code segments, among different programs, and across several memory devices. Similarly, operational data may be identified and illustrated herein within components, and may be embodied in any suitable form and organized within any suitable type of data structure. The operational data may be collected as a single data set, or may be distributed over different locations including over different storage devices, and may exist, at least partially, merely as electronic signals on a system or network. The components may be passive or active, including agents operable to perform desired functions.

Reference throughout this specification to “an example” means that a particular feature, structure, or characteristic described in connection with the example is included in at least one embodiment of the present disclosure. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present disclosure may be referred to herein along with alternatives for the various components thereof. It is understood that such embodiments, examples, and alternatives are not to be construed as de facto equivalents of one another, but are to be considered as separate and autonomous representations of the present disclosure.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive.

Those having skill in the art will appreciate that many changes may be made to the details of the above-described embodiments without departing from the underlying principles of the disclosure. The scope of the present disclosure should, therefore, be determined only by the following claims.

What is claimed is:

1. A method comprising:
obtaining information about a candidate record, wherein the candidate record includes information matching information of a target record, wherein the target record is stored in a database comprising a plurality of records and relationships between the records;
determining whether the candidate record comprises a greater number of relationships of a specific relationship type than the target record; and
marking one or more of the candidate record and the corresponding target record for review to indicate that a potentially new record for the database has been identified.

2. The method of claim 1, wherein obtaining information about the candidate record comprises receiving an indication that the candidate record may correspond to a same individual or entity as the target record.

3. The method of claim 1, wherein obtaining information about the candidate record comprises receiving the candidate record in response to a matching operation matching one or more values or parameters of the candidate record with the target record.

4. The method of claim 1, wherein the database comprises a genealogical database in which the plurality of records correspond to individuals and the relationships correspond to a family relation between the individuals corresponding to the records.

5. The method of claim 4, wherein the database comprises a family tree corresponding to a user or account.

6. The method of claim 4, wherein the specific relationship type comprises one or more of:
a parent relationship;
a child relationship; or
a marriage relationship.

7. The method of claim 1, wherein marking comprises storing and associating an identifier with the target record in the database, wherein the identifier indicates that a potentially new record not present in the database is available in the candidate record.

8. The method of claim 1, wherein marking comprises storing and associating information linking the candidate record with the target record in the database.

9. The method of claim 1, further comprising:
displaying a graphical hierarchical tree comprising nodes and connections between the nodes, wherein the nodes correspond to the plurality of records and the connections correspond to the relationships;
wherein marking the target record comprises graphically marking a node corresponding to the target record.

10. The method of claim 1, further comprising filtering a plurality of target records comprising the target record based on the presence of an indication that a potentially new record is in or associated with corresponding candidate records.

11. The method of claim 1, wherein the candidate record comprises one or more of:
a historical record corresponding to a historical document; or
a record in a second database separate from a first database, wherein the first database comprises the database.

12. A system comprising:
a candidate component configured to obtain information about a candidate record, wherein the candidate record includes information matching information of a target record, wherein the target record is stored in a database comprising a plurality of records and relationships between the records;
a relationship component configured to determine that the candidate record comprises a greater number of hierarchal relationships of a specific relationship type than the target record; and
a marking component configured to mark one or more of the candidate record and the target record for review to indicate that a potentially new record for the database has been identified.

13. The system of claim 12, wherein the candidate component is configured to obtain information about the candidate record by one or more of:
receiving an indication that the candidate record likely corresponds to a same individual as the target record; or
receiving the candidate record in response to a matching operation matching one or more values or parameters of the candidate record with the target record.

14. The system of claim 12, wherein the database comprises a genealogical database and wherein the plurality of records correspond to individuals and wherein the relationships correspond to a family relation between the individuals.

15. The system of claim 14, wherein the specific relationship type comprises one or more of a parent relationship, a child relationship, or a marriage relationship.

16. The system of claim 12, wherein the marking component is configured to mark one or more of the candidate record and the corresponding target record by storing and associating an identifier with the target record in the database, wherein the identifier indicates that information about a new individual not present in the database is likely available in the candidate record.

17. The system of claim 12, further comprising a display component configured to:
display a graphical hierarchical tree comprising nodes and connections between the nodes, wherein the nodes correspond to the plurality of records and the connections correspond to the relationships;
wherein the display component displays a node corresponding to the target record with a different color, shape, or other indicator than nodes without a corresponding candidate record with a greater number of relationships.

18. Computer readable storage media storing instructions that, when executed by one or more processors, cause the processors to:
obtain information about a candidate record, wherein the candidate record includes information matching information of a target record, wherein the target record is stored in a database comprising a plurality of records and relationships between the records, wherein the database comprises a genealogical database in which the plurality of records correspond to individuals and the relationships correspond to a family relation between the individuals;
determine whether the candidate record comprises a greater number of hierarchal relationships of a specific relationship type than the target record, wherein the specific relationship type comprises one or more of a parent relationship, a child relationship, or a marriage relationship; and
mark one or more of the candidate record and the target record as part of a class for which a potentially new record for the database has been identified.

19. The computer readable storage media of claim 1, wherein the instructions are configured to cause the processors to mark one or more of the candidate record and the target record by storing and associating an identifier with the target record in the database, wherein the identifier indicates that a potentially new record not present in the database is available in the candidate record.

20. The computer readable storage media of claim 1, further comprising instructions that cause the processors to:
displaying a graphical hierarchical tree comprises nodes and connections between the nodes, wherein the nodes correspond to the plurality of records and the connections correspond to the relationships;
wherein marking the target record comprises graphically marking a node corresponding to the target record.
